(19) United States
(12) Patent Application Publication
(10) Pub. No.: US 2019/0218591 A1
(43) Pub. Date: Jul. 18, 2019

(54) SYSTEMS AND METHODS FOR SCHEDULING AND SEQUENCING AUTOMATED TESTING PROCEDURES

(71) Applicant: SELUX DIAGNOSTICS, INC., Charlestown, MA (US)

(72) Inventors: Aleksandar Vacic, Charlestown, MA (US); Nathan Purmort, Charlestown, MA (US); Derek Puff, JR., Charlestown, MA (US); Frederick P. Floyd, JR., Charlestown, MA (US)

(21) Appl. No.: 16/245,990

(22) Filed: Jan. 11, 2019

Related U.S. Application Data
(60) Provisional application No. 62/617,163, filed on Jan. 12, 2018.
(60) Provisional application No. 62/662,592, filed on Apr. 25, 2018.

(51) Int. Cl. C12Q 1/18; C12M 3/00; G01N 35/04; G01N 35/00

(52) U.S. Cl. CPC C12Q 1/18; C12M 23/42; G01N 35/04; G01N 2035/0432; G01N 2035/0463; G01N 2035/00495; G01N 35/0092

(57) ABSTRACT

Automated Antimicrobial Susceptibility Testing (AST) systems and methods are provided in which samples are passed through one or more recursive or non-deterministic operations and are batched or re-batched to optimize the utilization of resources for subsequent deterministic operation sequences. Automated AST systems are also provided in which deterministic and non-deterministic workflows are spatially segregated.

### PRIORITY

This application claims the benefit of priority under 35 USC § 119 to United States Provisional Patent Application Ser. No. 62/617,163, filed Jan. 12, 2018 and 62/662,592, filed Apr. 25, 2018. Each of these applications are incorporated by reference herein in their entirety and for all purposes.

### FIELD OF THE DISCLOSURE

The present disclosure is related to in vitro diagnostic devices, systems, and methods, particularly microbiological diagnostic devices. The systems and methods described herein can relate to automated incubation and processing of microbiological samples.

### BACKGROUND

Antimicrobials have transformed the practice of medicine, making once lethal infections more easily treatable and saving millions of lives. Quick administration of antimicrobials has been proven to reduce mortality, especially in severe infections such as septicemia. In these severe cases, highly potent, broad-spectrum antimicrobials are most often used since information about organism (e.g., species) is typically not known. These broad-spectrum antimicrobials can have serious side effects, cause organ damage, prolong recovery and hospital stays, and in some cases increase mortality. Furthermore, the overuse of antimicrobials has caused the rise of antimicrobial resistant organisms, which have become a serious and growing threat to public health. A growing body of evidence suggests that, by using targeted antimicrobial therapy, patient mortality can be reduced (e.g., minimized), recovery can be shortened, and hospitals can save money on both patient stay and minimizing usage of expensive antimicrobials.

However, complete information typically needed for targeted antimicrobial therapy is typically delivered 2-3 days after a sample is taken. Current antimicrobial susceptibility tests (AST) may require more than 8 hours to determine and deliver relevant and useful information, which is typically not sufficient to provide a same day result. Because many clinical laboratories operate on 12-hour shifts, this means that actionable AST information is not available to prescribers until the following day.

Some systems perform phenotypic AST testing of patient samples by exposing them to a set of antimicrobial dilution series and measuring their growth over time. Growth can be measured indirectly and most frequently optically by measuring solution turbidity or fluorescence of a dye triggered by microorganism metabolism. By quantitative comparison of optical signal, these systems determine the lowest concentration in dilution series of each antimicrobial that successfully inhibits growth of the tested microorganism. This value, known as minimum inhibitory concentration (MIC), is often used by clinicians to determine the most effective antimicrobial and dosage, i.e., to deliver targeted antimicrobial therapy. In addition, a qualitative susceptibility result (QSR) of susceptible (S), intermediate (I) or resistant (R) may be reported with or instead of MIC.

To arrive at MIC and QSR results, the growth of a given microorganism in standardized nutrient broth (e.g., Muller Hinton broth) is compared to its growth in multiple antimicrobial dilution conditions (e.g., across a 2× dilution series). Manually, growth is typically measured only once, after 16-24 hours, as defined by Clinical & Laboratory Standards Institute (CLSI). Some automated systems, as previously mentioned, shorten this time by interrogating microorganism growth in each test well periodically (e.g., 20 minutes). This process can be tedious and is typically not performed by technicians. Growth curves are then analyzed using proprietary algorithms that include analysis of absolute values, relative values between wells, rates, integrals, etc., of growth curves.

Historically, automation in microbiology clinical laboratories has lagged compared to clinical chemistry and hematology areas where automation and new assay development have reduced time from sample to result. Three major commercial automated AST systems have been developed in the past 30 years; all were designed to automate operations typically done by highly trained technicians. These automated systems perform operations that are superficially similar to the operations performed by automated systems developed in the fields of immunoassay, nucleic acid testing, cytology, etc. (e.g., sample incubation, fluid handling, and so on). However, phenotypic AST applications require these operations to be performed under conditions that are often not compatible with the design limitations of existing automated devices.

### SUMMARY

This disclosure provides systems and methods for streamlining process flows in automated AST systems, maximizing throughput and minimizing time to result.

In one aspect, the disclosure relates to an automated AST system that includes two portions: a first portion configured to perform a recursive operation (e.g., incubation of a sample until growth of a microbe therewithin is sufficient for performance of an AST testing sequence) and a second portion spatially separated from the first portion and configured to perform a fixed sequence of operations on the AST cassette (e.g., the AST testing sequence). The system may include an assembly for moving AST cassettes between the first and second portions, such as an elevator in those instances where the first and second portions of the system are vertically separated; or for horizontally separated systems, an incubator open at two ends, each opening accessible from a portion of the system (e.g., a first opening accessible from the first portion of the system, a second opening accessible from the second portion of the system). In some embodiments, the system includes a gantry and effector arm for moving AST cassettes between the incubator assembly and an assembly for measuring an optical signal.

Continuing with this aspect of the invention, in some embodiments the recursive operation assessment of microbial growth in at least one well of the AST cassette until a predetermined level of microbial growth is reached. That operation can comprise (a) incubating the AST cassette in an incubator assembly under conditions suitable for microbial growth, and (b) measuring an optical signal in at least one well of the AST cassette that is correlated with microbial growth. In these cases, the automated system optionally includes a two-axis platform with an extendable platter operating in a third axis or an arm with an effector for moving the AST cassette between the incubator assembly and an assembly for measuring the optical signal. The fixed sequence of operations comprises, in some cases, contacting a microbe in each of a plurality of wells with a surface-binding reagent and separating the microbes from unbound surface-binding reagent. In a subset of these cases, the fixed sequence of operations is different for different AST cassettes or regions within an individual AST cassette.

In another aspect, this disclosure relates to a method of performing an antimicrobial susceptibility testing (AST) sequence comprising inserting, into an automated AST system, a plurality of AST cassettes, then incubating the plurality of AST cassettes for a first pre-determined interval under conditions suitable for microbial growth; recursively assessing a level of microbial growth (e.g., by means of a metabolic assay and/or an optical measurement) in at least one well of each AST cassette until the level of microbial growth exceeds a predetermined threshold, then releasing the AST cassette; grouping one or more AST cassettes released from the recursive assessment step into a multi-plate batch of AST cassettes; and subjecting the multi-plate batch of AST cassettes to a deterministic processing sequence utilizing a limited-access resource, optionally a centrifuge, a fluid handling station, or an optical signal measurement station). In some cases, the deterministic processing sequence comprises, for a plurality of wells in each AST cassette in the multi-plate batch of AST cassettes: contacting a microbe in the well with a solution comprising a surface-binding reagent; separating the microbe from the solution comprising the surface binding reagent; rinsing, at least once, the microbe in a solution that does not comprise the surface binding reagent; assessing a level of surface-binding reagent present in the well; and for each AST cassette, based on a comparison of surface binding reagent present in different wells of the AST cassette, determining at least one of a qualitative susceptibility result (QSR) or minimum inhibitory concentration (MIC) for an antimicrobial agent present in differing amounts in the plurality of wells of the AST cassette and reporting said QSR or MIC to a user of the automated AST system. In some instances, the step of recursively assessing a level of microbial growth in at least one well of each AST cassette comprises incubating each AST cassette not released into the multi-plate batch of AST cassettes for a second pre-determined interval which is shorter than the first pre-determined interval. The step of separating the microbe from the solution comprising the surface binding reagent can include centrifuging the AST cassette in a centrifuge. In some cases, an operation performed by the access-limited resource requires an even number of AST cassettes to be loaded into the access-limited resource, and optionally the access-limited resource is loaded with first and second multi-plate batches which are at different stages of the deterministic processing sequence.

Continuing with this aspect of this disclosure, in some embodiments the deterministic processing sequence is organized into a plurality of operation cycles, and wherein the movement of multi-plate batches into and out the access-limited resource during the deterministic processing sequence according to a pipeline sequence that minimizes the number of operation cycles during the deterministic processing sequence in which the access-limited resource is idle. In some cases, for instance, a deterministic processing sequencer controls a rate at which new multi-plate batches enter the deterministic processing sequence based the pipeline sequence and, optionally, based on an input from a batch aggregator pipeline sequence. The deterministic processing sequence in these embodiments may be divided into a plurality of subsequences, each of which optionally includes one or more tasks that are executed in parallel on the same station of the instrument when a plurality of multi-plate batches are running in the deterministic processing sequence. Alternatively, the method may be implemented such that the subsequences are not performed concurrently for a single multi-plate batch.

In some embodiments according to this aspect of the disclosure, a size of the multi-plate batch of AST cassettes is defined before the batch is subjected to the deterministic processing sequence based on a number of cassettes being processed by the automated AST system, and wherein the maximum batch size increases from one plate to multiple (e.g. 2, 4, 6, 8, 10, 12) plates. Some embodiments are characterized by one or more of the following: the limited-access resource is a centrifuge sized to centrifuge up to 6, 10, 12, 14 or 16 AST cassettes at a time; the plurality of AST cassettes are grouped into single-plate batches during the steps of incubating the plurality of AST cassettes and recursively assessing a level of microbial growth; the step of inserting the plurality of AST cassettes comprises inserting at least AST cassettes into the automated AST system at a rate of up to 4 plates every 12 minutes; and/or on average, no more than 8 hours elapses between the step of inserting an AST cassette into the system and the step of reporting the QSR or MIC to a user of the automated AST system for the same AST cassette, or no more than 6 hours.

### BRIEF DESCRIPTION OF THE DRAWINGS

FIG. 1 depicts exemplary process flows according to embodiments of this disclosure.

FIG. 2 depicts an exemplary process flow according to embodiments of this disclosure.

FIG. 3 depicts sample queueing and re-queuing by a batch aggregator according to certain embodiments of this disclosure.

FIG. 4 depicts an exemplary process flow according to embodiments of this disclosure.

FIG. 5 depicts an exemplary process flow according to some embodiments of this disclosure.

FIGS. 6A, 6B, 6C and 6D depict exemplary automated AST systems according to the present disclosure.

FIG. 7 depicts an optimally scheduled workflow for an automated AST system.

FIG. 8 depicts an optimally scheduled workflow for an automated AST system.

### DETAILED DESCRIPTION

### Overview

Throughout this disclosure, reference is made to samples, series and jobs. The term sample refers to any sample processed by a system or method of this disclosure in any suitable format, including a multi-well plate or cartridge format. The term series refers to a sequence of process steps performed by a system of this disclosure on an individual sample. Each set of series for an individual sample is referred to as job. Reference is also made to sample plates, AST panels, AST cassettes, and/or AST cartridges. These terms are used interchangeably to refer to multi-well vessels that are inoculated with patient samples and processed by automated AST systems according to various embodiments of the following disclosure.

Selux Diagnostics (Boston, Mass.) has recently developed automated AST systems that can yield rapid phenotypic AST results based on one or more endpoint assays rather than the growth curves used by currently available commercial systems. The operation of these systems is described in U.S. Pat. No. 10,161,948 to Vacic et al. which is incorporated by reference herein. One challenge in implementing such systems in clinical laboratories relates to the integration of deterministic operations—e.g., media exchanges, optical interrogation and like operations which are performed in fixed sequence and/or at determined times—with non-deterministic and/or recursive operations, such as checkpoints for sufficient microbial growth.

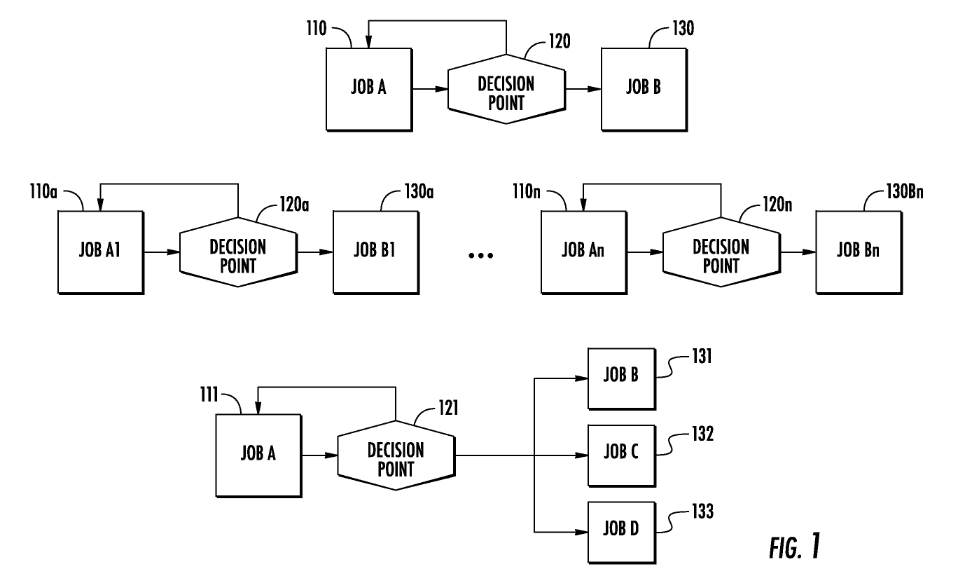

Referring to FIG. 1, in some embodiments, a single process flow according to the present disclosure can be logically divided into groups of non-deterministic sub-processes 110 (Job A) and deterministic sub-processes 130 (Job B) separated by a decision point 120. In some instances, a process flow comprises multiple parallel non-deterministic processes 110a-110n and multiple parallel deterministic processes 130a-130n, each separated by multiple respective decision points 120a-120n. Alternatively, a process flow according to this disclosure may comprise a non-deterministic process (Job A) 111, a Decision point 121 and a plurality of operations 131, 132, 133, that can be performed after Decision Point 121. Process flows can be performed on individual samples or may be batched on either side of the decision point.

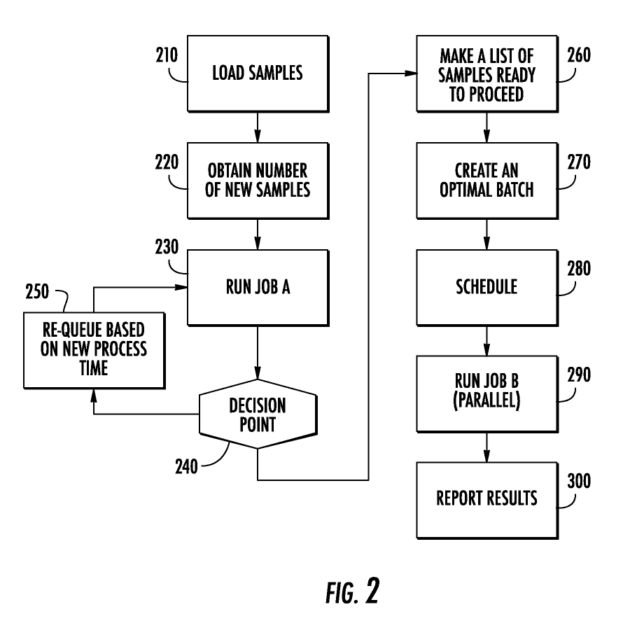

Referring to FIG. 2, in one type of workflow according to this disclosure, samples are loaded into a device 210 (e.g., an antimicrobial susceptibility testing system, such as those described below). The number of samples is obtained 220 and the samples are queued sequentially or scheduled in batches for non-deterministic sub-processes (Job A) and stored in a data structure (e.g., array, list). After running Job A 230 and passing through a decision point 240 (e.g., optical interrogation of the sample under test) the queue of samples can be sorted based on their readiness to be released from the current operation either re-queued 250 into Job A 230, or released to deterministic sub-process 260. After a list of released samples is made, the samples are placed into an optimal batch 270 and scheduled 280 for Job B 290. Following completion of the deterministic sub-processes, results of the analysis are reported 300.

In the workflows described herein, samples can be queued following any non-deterministic portion of the process. At a given period (e.g., every 2, 5, 10, 20, 30, 60 minutes, or every 1, 2, 3, 4, 5, 6, 7, 8 hours), based on process needs, batches of samples are released from the queue into a deterministic portion of the process.

If a non-deterministic sequence of sub processes follows a deterministic sequence, batches are broken into individual samples again (or the most logical batches) and can be processed independently through the non-deterministic sequence. This flow of making and breaking batches for deterministic and non-deterministic portions of the process can be continued until the process is complete for all samples.

The scheduling can be performed based on one or more job shop optimization algorithms such as, but not limited to, shortest processing time, shortest idle time, heuristic rules etc. Similarly, Job A may be performed in parallel for multiple samples. In this case the first scheduling will occur prior to start of Job A with optimal batches determined by the scheduling algorithm and system design. At each decision point the sample list is sorted and appended with new samples. Samples ready for release are batched and scheduled in Job B queue.

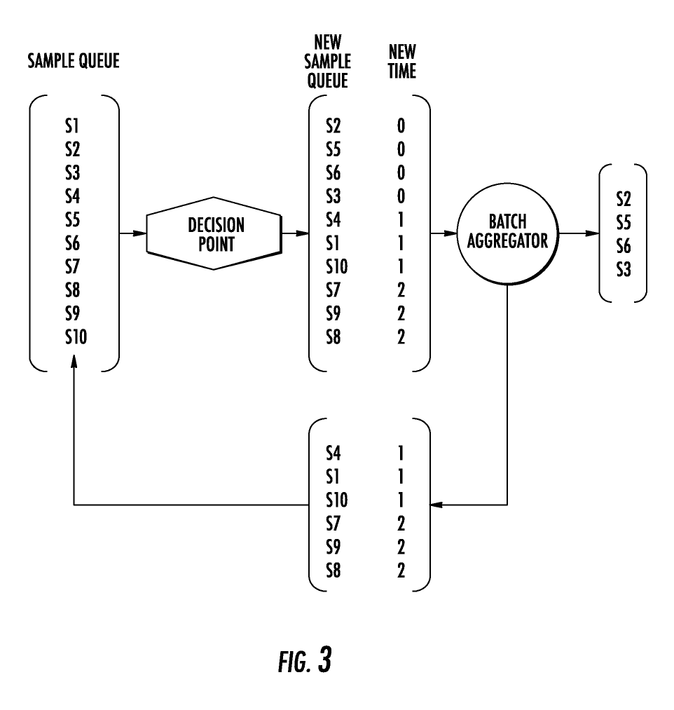

The example flow chart in FIG. 3 depicts an exemplary scheduling process described herein. At t=0, a number of plates (e.g., 10 plates) are loaded into the system. At t=T after decision point, in this example, it is determined that samples S2, S5, S6 and S3 are ready for next job, while samples S4, S1 and S10 will be (or are expected to be) ready at t=T+1 and samples S7, S9 and S8 will be (or are expected to be) ready in t=T+2. A batch aggregator will release the first group of plates and schedule them for Job B, while groups 2 and 3 will be returned into original queue. If additional plates (e.g., S11, S12, S13 and S14) are added into the system, the new queue will look like (S4, S1, S10, S7, S9, S8, S11, S12, S13, S14). At the next decision point, another requeuing may occur based on obtained information for S11, S12, S13 and S14 etc.

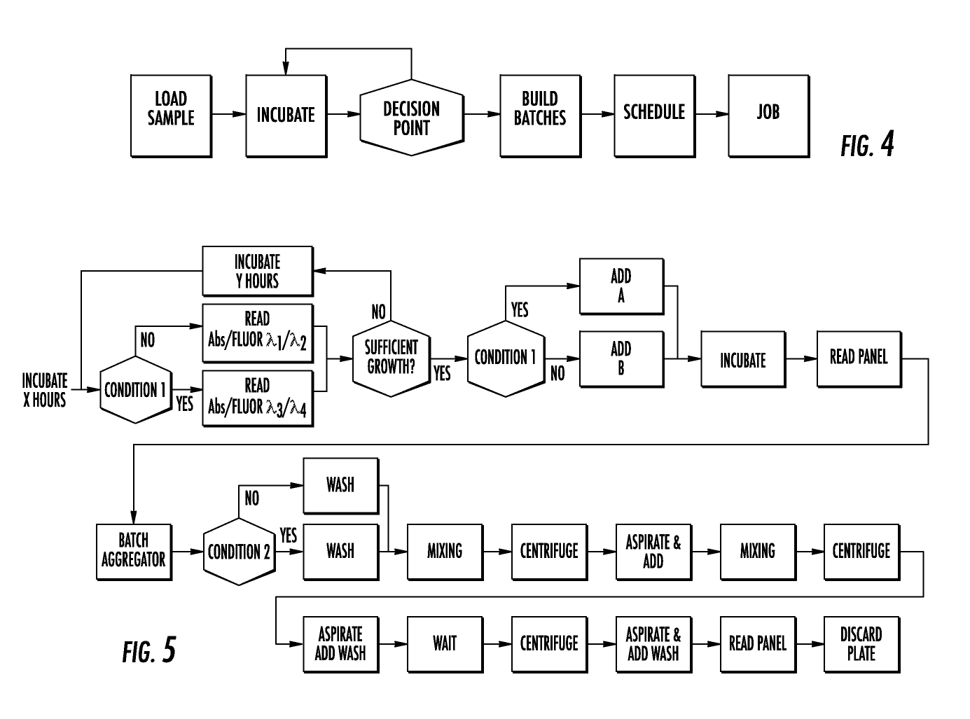

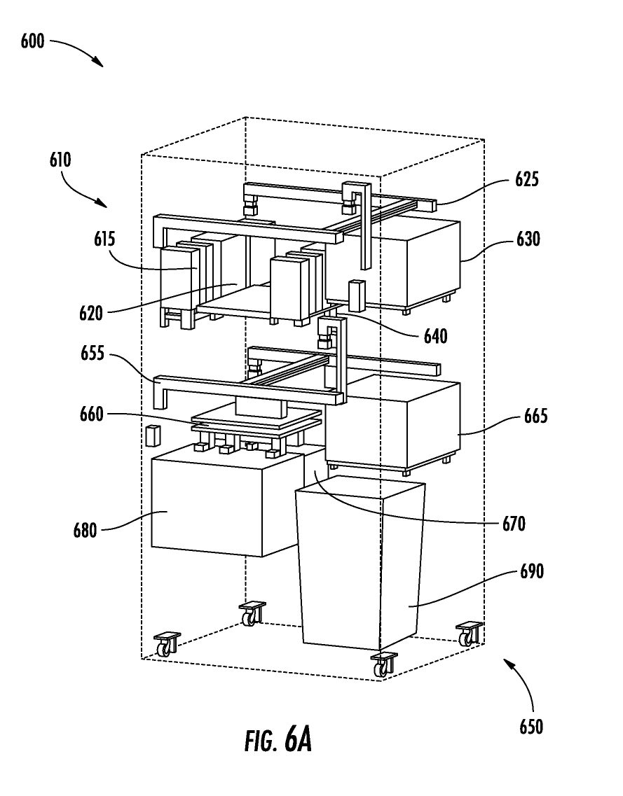

FIG. 4 shows a generic assay with a non-deterministic first half gated by a time-dependent check point. After the checkpoint, samples are re-batched, optimally scheduled and send for processing. FIG. 5 illustrates an example of an antimicrobial susceptibility assay based on a surface binding endpoint assay, preceded by a sufficient growth check and metabolic assays. The sufficient growth assay allows indicator dye-free incubation of bacteria and antibiotics similar to a manual broth microdilution assay. The absence of dye allows reduced (e.g., minimal) interference of the said dye on bacterial growth (i.e. metabolism). Only a few control wells may contain the dye for monitoring bacterial growth. In another embodiment, growth can be measured dye-free using absorbance, forward or backward light scattering or bacterial autofluorescence. This part of the assay is non-deterministic at the time of plate loading and corresponds to Job A in FIGS. 1-4. This process can be sequential or may use some logical batching if hardware allows so. Once sufficient growth is achieved, plates can be batched in a new set of logical batches and rescheduled to allow optimal throughput.

Even when the throughput optimization steps described above are implemented, those of skill in the art will appreciate that bottlenecks may occur in any process due to physical system constraints, among other factors. For instance, in an automated AST process as described above, samples which are re-batched may occupy incubation stations of an instrument, limiting processing of additional cartridges through the same stations. Bottlenecking may also occur due to the potential for physical interference between gantries and gripper arms moving samples based on non-deterministic operations. One mechanism to reduce such bottlenecking is to physically separate deterministic and non-deterministic operations, for example by placing stations involved in these operations on different sides or levels of an instrument. Vertical stratification of components may be particularly advantages, since a vertically stratified instrument will occupy less floor space than a non-stratified instrument, and floor space is at premium in many clinical laboratories.

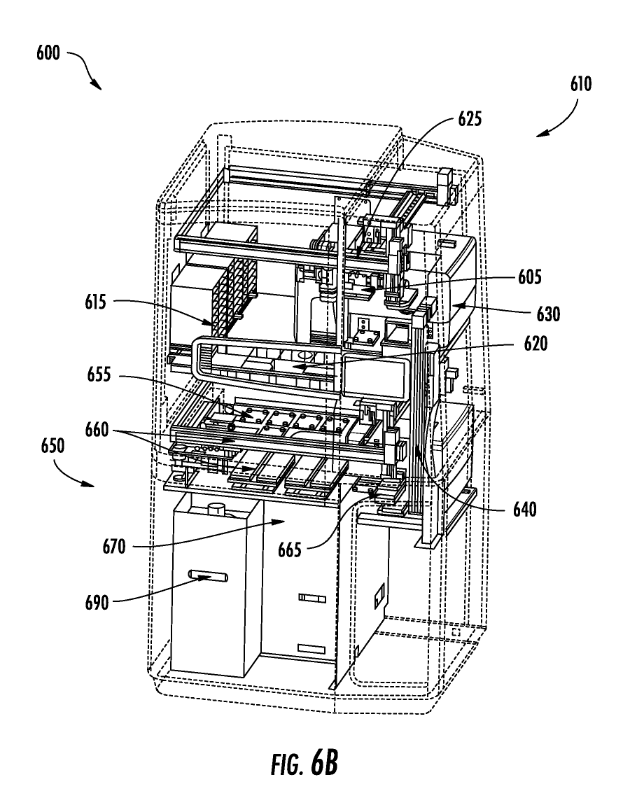

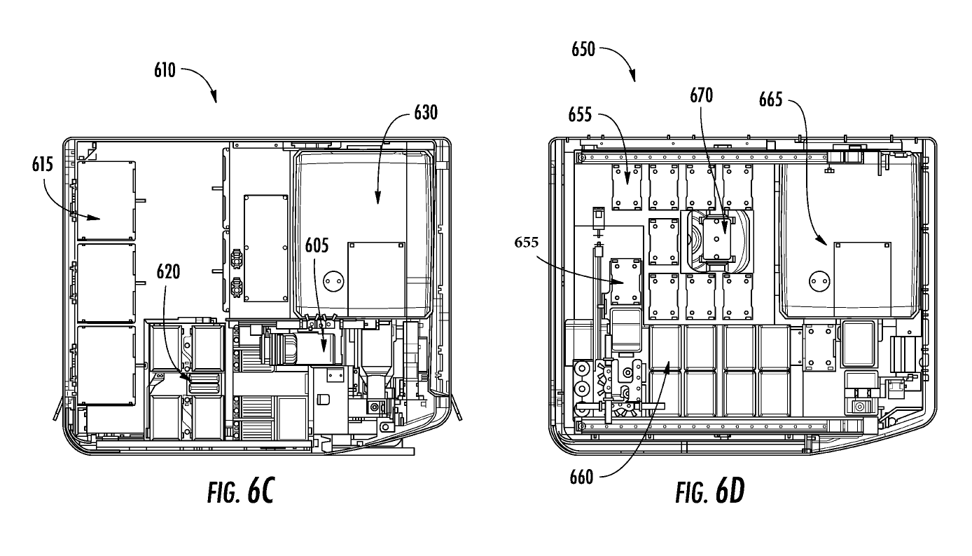

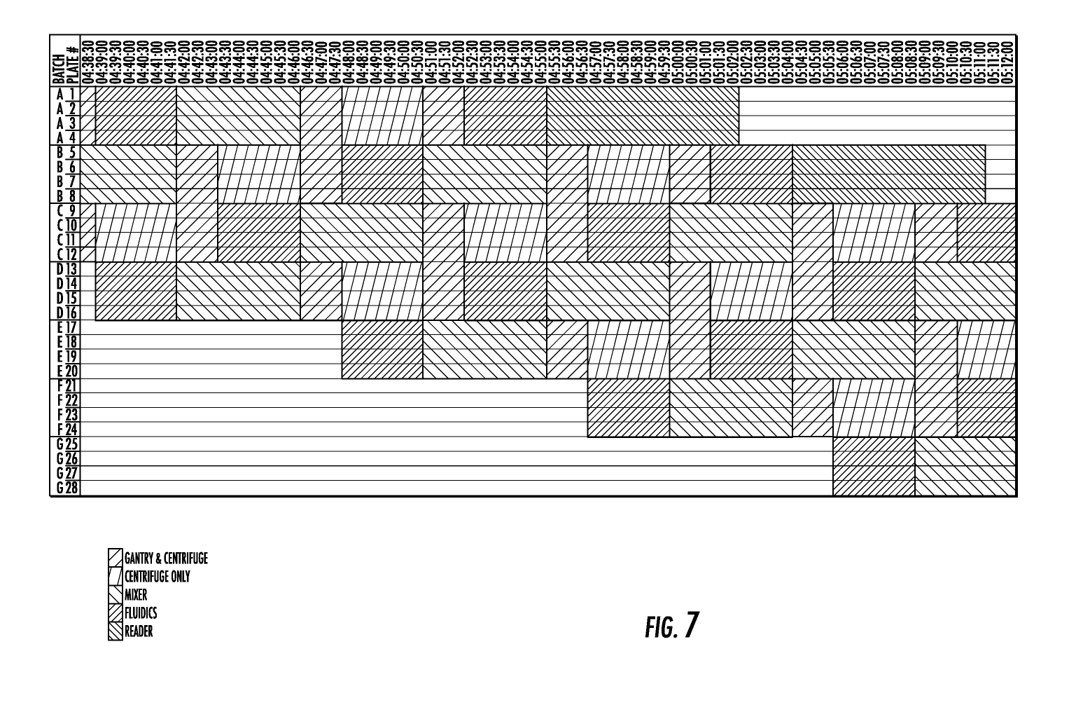

FIG. 6 shows one example of an automated AST system that segregates different operations of AST sample sequences while implementing one or more scheduling approaches described herein. For example, the sample scheduling approaches can include both deterministic and non-deterministic sequences of sub-processes based on an example workflow depicted in FIG. 5. System 600 is divided in two sub-systems or regions (e.g., floors), an upper floor 610 which includes stations dedicated to handling non-deterministic parts of the workflow, and a lower floor 650 which includes stations dedicated to handling the deterministic part of the workflow. The system 600 can include a sample loading area 620 where an operator inserts new samples for processing. In some cases, the operator can load one or more well plates, trays, or cartridges into the system. The upper floor 610 can include a gantry with an end-effector (e.g., gripper) 625, an optical reader 630, one or more incubators (e.g., shaker incubators) 615, and a fluid handling station or system 605. Once sufficient growth is achieved in the non-deterministic sub-process, plates can be queued into batches (e.g., batched for better (e.g., more efficient) sequencing and throughput) and send them to a lower floor for processing. In some cases, the plates can be sent to the lower floor using an elevator 640 (e.g., a vertical robotic device), which can serve as a hand-shake or synchronization tool between the two sub-systems on the two floors. Part of the assay between the Condition 1 and Batch Aggregator (depicted in FIG. 5) can take place on either floor. In some cases, due to the additional incubation, assigning that part of the assay to the upper floor can be more space efficient and can reduce repeated introduction and removal or samples from incubators on the lower floor. In some cases, such as where space is not an issue, that part of the assay can be assigned to the lower floor. In some cases, the control of the elevator 640 may be assigned to the floor that runs more complex process (e.g. bottom floor). This allows the incoming plate rate for that floor to be controlled to allow the most optimal steady state batching (e.g. the highest throughput) for that floor and thus the overall system.

Lower floor 650 is configured to group plates in efficient (e.g., optimal) batches and may include a gantry with an end-effector/gripper 655, a mixer and fluidics station 660, an optical reader 665, a sample separation station 670 (e.g., a centrifuge). The system 600 also includes fluid storage 680 and waste vessels 690. In some cases, wait stations 655 for holding plates between other stations act as buffers may be added to allow better utilization of those stations (e.g. gantries with grippers, mixers etc.).

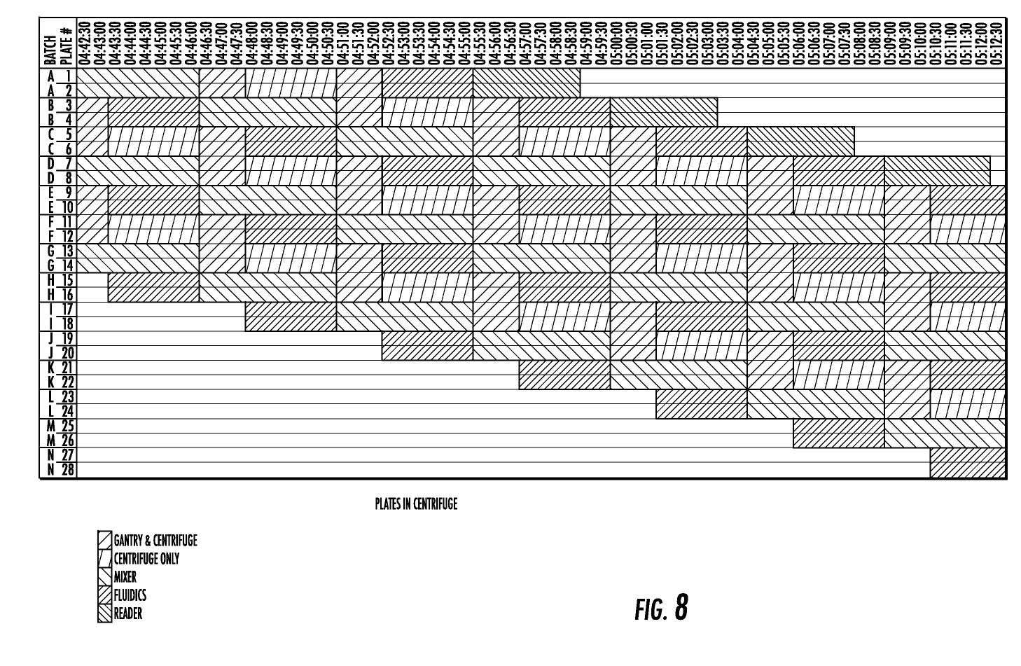

Batch size is one variable that can be particularly useful in optimizing a processing sequence for full utilization of limited-capacity resource such as a centrifuge. FIGS. 7 and 8 show example scheduling sequences for a centrifugation-based processing sequence. FIG. 7 illustrates the optimized scheduling of 4-plate batches, requiring a centrifuge with 8-plate capacity. FIG. 8 illustrates the same sequence optimized using 2-plate batches, requiring a centrifuge with only 6-plate capacity to achieve the same overall throughput. While not wishing to be bound by any theory, it is believed that because the centrifuge in the optimized sequence of FIG. 8 has the capacity to process up to three batches at a time, the effects of pipelining are more pronounced than they might be in another system in which the centrifuge (or other access-limited system resource) carries fewer batches. Additionally, and again without wishing to be bound by any theory, those of skill in the art will appreciate that the workflow depicted in FIG. 5 includes three centrifugation steps, and that by utilizing a centrifuge with a capacity of at least three batches, it may be possible to process batches in a highly pipelined fashion at all three stages of the process, improving utilization of the centrifuge (defined as operating time divided by total system time). Skilled artisans will also appreciate that optimal batch size is influenced by several factors, including the capacity of the access-limited resource (e.g., the centrifuge), but also the processing times of other subsystems,

### Automated Endpoint-Based AST Systems and Methods

The systems and methods described herein can relate to automated rapid antimicrobial susceptibility testing systems for performing multi-assay testing sequence, where the testing systems can be configured to at least: receive a loaded test plate; move the loaded test plate to an incubation assembly; incubate and agitate an inoculated sample within the test plate in the incubation assembly; at least once, periodically measure an amount of sample growth in a plurality of control wells of the test plate; responsive to determining that a level of growth in the control wells meets or exceeds a threshold level of growth, stop incubation; perform one or more end point assays on incubated samples in the test plate; measure one or more optical outputs from the sample in the plurality of wells of the test plate obtained from the said end-point assays, the optical output corresponding to an amount of the microorganism remaining in each of the plurality of wells; and report at least one of: a minimum inhibitory concentration of and/or a qualitative susceptibility interpretation for the microorganism remaining in each of the plurality of wells and the plurality of antimicrobials.

For example, in some embodiments, samples to be tested can be inoculated into a test panel (e.g., a cartridge (e.g., a test tray (e.g., a well plate (e.g., a microwell plate (e.g., a 96 or 384 microwell plate (e.g., microtiter plate)))))). In some cases, the cartridges are loaded into the system and can then be handled substantially automatically without human interaction (e.g., using robotics) until the end of the process. Process results can be reported, for example, on a display screen and communicated to a laboratory information system (LIS). Additionally, each cartridge can be uniquely defined by a barcode or other unique marking (e.g., laser engraving, direct part marking, RFID, or other marking/identification) that can be scanned either by a user prior to loading or automatically by the system to identify the cartridge and samples to be tested therein. Additionally, one AST panel (e.g. a microwell plate (e.g. a 96 or 384 microwell plate) may contain 2 or more clinical samples (e.g. a multiplexed panel).

In some embodiments, cartridges can include a plurality of test cartridge chambers (e.g., wells), each containing a liquid or dried form of an antimicrobial. In some cases, each well can contain a different antimicrobial type and/or concentration. In some cases, the cartridge can have the dried antimicrobials in the wells before the cartridge is loaded into the system. In some cases, the cartridge can have antimicrobials suspended in a medium (e.g., a fluid, such as nutrient broth, e.g., Mueller Hinton Broth). In some cases, the cartridge can have antimicrobials in the form of an antimicrobial film. In some cases, the cartridge can have antimicrobials in solid form. The cartridge can be inoculated with a sample containing microorganisms and loaded into the rapid AST diagnostic apparatus. The microorganisms described herein can be derived from biological samples. In some embodiments, the biological sample is derived from a clinical sample (e.g., which can originate from a patient sample). Example biological samples can include whole blood, plasma, serum, sputum, urine, stool, white blood cells, red blood cells, buffy coat, tears, mucus, saliva, semen, vaginal fluids, lymphatic fluid, amniotic fluid, spinal or cerebrospinal fluid, peritoneal effusions, pleural effusions, exudates, punctates, epithelial smears, biopsies, bone marrow samples, fluids from cysts or abscesses, synovial fluid, vitreous or aqueous humor, eye washes or aspirates, bronchoalveolar lavage, bronchial lavage, or pulmonary lavage, lung aspirates, and organs and tissues, including but not limited to, liver, spleen, kidney, lung, intestine, brain, heart, muscle, pancreas, and the like, swabs (including, without limitation, wound swabs, buccal swabs, throat swabs, nasal swabs, vaginal swabs, urethral swabs, cervical swabs, rectal swabs, lesion swabs, abscess swabs, nasopharyngeal swabs, and the like), and any combination thereof. Also included are bacteria cultures or bacteria isolates, fungal cultures or fungal isolates. In some cases, one or more dilution, isolation, and/or culturing steps can be performed prior to microorganism inoculation.

In some embodiments, prior to loading the automated AST system, the cartridge can be preheated to a temperature that corresponds to the desired temperature of incubation. Preheating can be useful in some cases since standard air convection incubators typically take 30 to 60 minutes to bring a test plate to a desired working temperature. Preheating can be particularly useful for use with the systems and methods described herein for performing rapid AST since typical desired incubation times are below 8 hours and in most cases less than 7 hours, less than 6 hours, less than 5 hours, less than 4 hours, or less than 3 hours. In some embodiments, the incubation of the microorganisms in the presence of the one or more antimicrobials occurs within 30 minutes after preheating the cartridge.

In some embodiments, the plurality of liquid-containing wells in the cartridge can be preheated to a temperature that is from about 30 degrees C. to about 40 degrees C. In some cases, the preheating can substantially uniformly heat the wells of the cartridge. In some embodiments, the substantially uniform heating of the wells can include heating the cartridge so that a percent difference of temperature between a highest-temperature well on a cartridge and a lowest-temperature well on the cartridge that is less than about 5%. That is, in some embodiments, a variation of temperature across the cartridge (e.g., from well to well) is less than about 5%. In some cases, the cartridge is preheated by an addition of at least one fluid at a temperature of at least about 25 degrees C. to the cartridge.

In some embodiments, the cartridge can be preheated for less than about 15 minutes. In some cases, the cartridge is preheated for about 1 minute, about 2 minutes, about 5 minutes, about 10 minutes, or about 15 minutes. In some embodiments, the cartridge is preheated by at least one of radiative heating, conduction heating, and/or convection heating. For example, radiative heating can include infrared radiative heating. In some examples, the cartridge can be preheated by conduction and convection heating. For example, at least one heating surface can perform the conduction and convection heating. In some embodiments, the cartridge can be preheated by both radiative heating and conduction and convection heating. In some embodiments, the cartridge is not preheated by convection heating alone.

In some embodiments, the systems and methods herein provide for automation of a rapid AST, from an inoculated cartridge loading by a technician to a result (e.g., minimum inhibitory concentration and CLSI breakpoint interpretation). In some cases, the cartridge is loaded by a technician and organism identification (ID) information (e.g., species), such as Staphylococcus aureus, can be entered or can be obtained automatically by the system's software interface. In this way, ID information obtained using other methods such as mass spectrometry (e.g., MALDI-TOF), nucleic-acid hybridization-based detection (e.g., fluorescent in-situ hybridization or FISH) and/or multiplex polymerase chain reaction (PCR) testing can be utilized. In some cases, a cartridge with colorimetric and fluorometric dyes can be used for microorganism ID, known to those skilled in the art as biochemical testing.

In some embodiments, the systems described herein can incubate cartridges and, after a defined period of time (e.g., at least 2 hours) after a cartridge is loaded into the system, interrogate growth check wells at a single timepoint or periodically to perform the checkpoint assay. Once sufficient growth of the sample in the growth check well is detected, the systems described herein can initiate end point assays. The checkpoint assay typically involves direct (e.g., absorbance, nephelometry) or indirect optical measurements (e.g., florescence readout of a metabolic dye) of growth (using a microorganism in nutrient broth) to no growth (using nutrient broth without microorganisms therein) and/or “frozen-in-time” (FIT) control (measuring the growth or no growth control wells relative to another control well with microorganisms in non-nutritive media such as saline). Indirect measurements can include fluorometric measurements of wells where a reporter can be a redox dye that is converted into a fluorescent form via microorganism metabolism (e.g., resazurin). In such cases, the more microorganisms that are present in a well, the larger the amount of dye converted to fluorescent form, and thus a higher level of fluorescence is measured. That is, the more microorganisms that are present in a well, the faster the conversion to fluorescent form resulting in higher concentration of fluorescent product, and thus a higher level of fluorescence can be measured. In some embodiments, pH sensitive dyes (e.g., phenol red) can be utilized.

Upon determining that sufficient growth of the sample in the growth well is achieved, the systems and methods described herein can initiate one or more endpoint assays. The endpoint assays can include one or more liquid handling (e.g. addition of the amplifier designed to bind to microorganism), sample separation (e.g., centrifugation, magnetic separation, or vacuum filtration), and aspiration steps during which any unbound amplifier, bound to the surface of the microorganism in previous liquid addition steps, can be washed away, and finally an optical signal can be measured and correlated to antimicrobial dilutions and MIC and/or QSR can be determined. Multiple endpoint assays can be performed in the same wells and/or in different wells. Multiple endpoint assays may be advantageous for obtaining accurate MIC and/or QSR data.

In a final step, a time gated luminescence (e.g., time resolved fluorescence) can be utilized to measure an optical signal from the amplifier. In some cases, methods can allow excitation of an amplifier molecule and detection of emitted light, which can be separated both temporally (e.g., detection can be delayed and occurs after excitation when all auto fluorescence has died out) and spectrally (e.g., wavelength of excitation can be more than 100 nanometers (nm) apart from emission which allows usage of less expensive band pass filters). In some embodiments, amplification can be achieved by the addition of a substrate that is catalytically modified by the bound molecule and optical output can be measured. This optical signal can include absorbance signals, fluorescence signals, and/or chemiluminescence signals. In some embodiments, the signal can include electrochemiluminescence (ECL). In some embodiments, upconverting nanoparticles can be used as reporter molecules.

Endpoint assays that may be performed by the system include, but are not limited to, the following: a metabolic assay, a surface-binding probe assay, a chemical probe assay, a biochemical probe assay, an ATP assay, a nucleic acid probe assay, a double-stranded nucleic acid probe assay, an optical density assay, a visual assay, and a pH molecular probe assay.

In some embodiments, a separation (e.g., centrifugation) step is used to separate unbound amplifier from microorganism surface. Centrifugation utilizes differences in densities of microorganisms and surrounding fluid to create microorganism pellet. As one skilled in the art would appreciate, these separation methods use relative centrifugal forces (RCF) of 100 to 20,000 g's (where g is Earth's gravitational acceleration) can be used. The larger the RCF, the shorter the time for separation is typically needed. Typical ceiling value (e.g., the highest expected reasonable value) for cartridges, such as 96- or 384-microwell plates, is about 5,000 g to reduce the likelihood of the cartridges physically degrading, for example, chipping or breaking. In some embodiments, the centrifugation subsystem can generate the desired relative centrifugal forces generated in the centrifugation systems that can be from about 2,000 g to about 5,000 g (e.g., about 2,500 g to about 4,000 g (e.g., about 2,500 g)). In some embodiments, the centrifugation can be performed for at least 2.5 minutes. In some cases, the 2.5 minutes can include the time to achieve the desired centrifugation speed (e.g., ramp-up time), which can be, for example, about 45 seconds. Configuring the separation system to be a centrifugation system design can allow samples (e.g., the cartridge) to be accessed more easily by a robotic gripper to load and unload cartridges. This helps to allow full automation of sample processing. In some embodiments, the centrifugation system can be configured to accommodate multiple plates per centrifuge rotor position (e.g., by stacking plates on each other). Such stacking permits simultaneous centrifugation of at least 4 cartridges and up to 16 cartridges in a 4-position centrifuge. In some cases, if an odd number of cartridges are being processed, one or more ballast plates can be used to balance the centrifuge. Commercially available centrifuges, which can be compatible with robotic loaders, that can be modified to be compatible and implemented with the testing systems described herein are made by Hettich Lab Technology of Beverly, Mass. and Tuttlingen, Germany, US; BioNex Solutions, Inc. of San Jose, Calif., US, and Agilent Technologies of Santa Clara, Calif., US.

Alternatively or additionally, as discussed herein, in some embodiments, pelleting of microorganisms can be accomplished using magnetic separation. For example, magnetic particles, which can be nanometer and/or micrometer size, with appropriate surface functionalization can be added to bind to microorganism surface. Binding of end point assay can be done simultaneously with magnetic particle binding (competitive assay) or after magnetic capture (either as binding to a pellet or after resuspension in solution). In some cases, magnetic capture can be retractable to allow resuspension and can be incorporated in a stand that allows orbital or axial agitation. In some cases, separation can be done using filtration, e.g., vacuum filtration. In other cases, separation may not be necessary to separate unbound probe.

The foregoing disclosure has focused on a handful of exemplary workflows and systems, but those of skill in the art will appreciate that certain modifications can be made without departing from the spirit of the disclosure. Those skilled in the art will recognize, or be able to ascertain using no more than routine experimentation, certain equivalents to the specific embodiments described herein. Such equivalents are intended to be encompassed by the following claims.

1. An automated AST system, comprising:
a first portion configured to perform a recursive operation on an AST cassette; and
a second portion configured to perform a fixed sequence of operations on the AST cassette, wherein the second portion is spatially separated from the first portion.

2. The automated AST system of claim 1, further comprising an assembly for moving AST cassettes between the first and second portions.

3. The automated AST system of claim 2, wherein the first and second portions are separated vertically and the assembly for moving AST cassettes between the first and second portions comprises an elevator.

4. The automated AST system of claim 3, further comprising a gantry and effector arm for moving the AST cassette between the incubator assembly and an assembly for measuring an optical signal.

5. The automated AST system of claim 2 wherein the first and second portions are separated horizontally and the assembly for moving AST cassettes between the first and second portions comprises an incubator accessible from two ends—each end facing a portion of the system.

6. The automated AST system of claim 1, wherein the recursive operation is the assessment of microbial growth in at least one well of the AST cassette until a predetermined level of microbial growth is reached.

7. The automated AST system of claim 6, wherein the assessment of microbial growth comprises (a) incubating the AST cassette in an incubator assembly under conditions suitable for microbial growth, and (b) measuring an optical signal in at least one well of the AST cassette that is correlated with microbial growth.

8. The automated AST system of claim 6, further comprising one of (a) a two-axis platform with an extendable platter operating in a third axis or (b) an arm comprising an end effector, for moving the AST cassette between the incubator assembly and an assembly for measuring the optical signal.

9. The automated AST system of claim 1, wherein the fixed sequence of operations comprises contacting a microbe in each of a plurality of wells with a surface-binding reagent, waiting for the reagent to bind with or without agitation, and separating the microbes from unbound surface-binding reagent.

10. The automated AST system of claim 9, wherein the fixed sequence of operations is different for different AST cassettes or regions within an individual AST cassette.

11. A method of performing an antimicrobial susceptibility testing (AST) sequence, comprising the steps of:
inserting, into an automated AST system, a plurality of AST cassettes
incubating the plurality of AST cassettes for a first pre-determined interval under conditions suitable for microbial growth;
recursively assessing a level of microbial growth in at least one well of each AST cassette until the level of microbial growth exceeds a predetermined threshold, then releasing the AST cassette;
grouping one or more AST cassettes released from the recursive assessment step into a multi-plate batch of AST cassettes; and
subjecting the multi-plate batch of AST cassettes to a deterministic processing sequence utilizing a limited-access resource, wherein the limited-access resource is optionally a centrifuge, a fluid handling station, or an optical signal measurement station).

12. The method of claim 11, wherein the deterministic processing sequence comprises, for a plurality of wells in each AST cassette in the multi-plate batch of AST cassettes:
contacting a microbe in the well with a solution comprising a surface-binding reagent and waiting for the reagent to bind with or without agitation;
separating the microbe from the solution comprising the surface binding reagent;
rinsing, at least once, the microbe in a solution that does not comprise the surface binding reagent;
assessing a level of surface-binding reagent present in the well; and
for each AST cassette, based on a comparison of surface binding reagent present in different wells of the AST cassette, determining at least one of a qualitative susceptibility result (QSR) or minimum inhibitory concentration (MIC) for an antimicrobial agent present in differing amounts in the plurality of wells of the AST cassette and reporting said QSR or MIC to a user of the automated AST system.

13. The method of claim 12, wherein the step of recursively assessing a level of microbial growth in at least one well of each AST cassette comprises incubating each AST cassette not released into the multi-plate batch of AST cassettes for a second pre-determined interval which is shorter than the first pre-determined interval.

14. The method of claim 12, wherein the step of separating the microbe from the solution comprising the surface binding reagent comprises centrifuging the AST cassette in a centrifuge.

15. The method of claim 12, wherein an operation performed by the access-limited resource requires an even number of AST cassettes to be loaded into the access-limited resource.

16. The method of claim 15, wherein the access-limited resource is loaded with first and second multi-plate batches and wherein the first and second multi-plate batches are at different stages of the deterministic processing sequence.

17. The method of claim 12, wherein the deterministic processing sequence is organized into a plurality of operation cycles, and wherein the movement of multi-plate batches into and out the access-limited resource during the deterministic processing sequence according to a pipeline sequence that minimizes the number of operation cycles during the deterministic processing sequence in which the access-limited resource is idle.

18. The method of claim 17, wherein a deterministic processing sequencer controls a rate at which new multi-plate batches enter the deterministic processing sequence based the pipeline sequence and, optionally, based on an input from a batch aggregator pipeline sequence.

19. The method of claim 18, wherein the deterministic processing sequence is divided into a plurality of subsequences.

20. The method of claim 19, wherein each subsequence in the plurality of subsequences includes one or more tasks that are executed in parallel when a plurality of multi-plate batches are running in the deterministic processing sequence.

21. The method of claim 19, wherein the subsequences are not performed concurrently for a single multi-plate batch.

22. The method of claim 11, wherein a size of the multi-plate batch of AST cassettes is defined before the batch is subjected to the deterministic processing sequence based on a number of cassettes being processed by the automated AST system, and wherein the maximum batch size increases from one plate to multiple (e.g. 2, 4, 6, 8, 10, 12) plates.

23. The method of claim 11, wherein the limited-access resource is centrifuge sized to centrifuge up to 6, 10, 12, 14 or 16 AST cassettes at a time.

24. The method of claim 11, wherein the plurality of AST cassettes are grouped into single-plate batches during the steps of incubating the plurality of AST cassettes and recursively assessing a level of microbial growth.

25. The method of claim 11, wherein the step of inserting the plurality of AST cassettes comprises inserting at least AST cassettes into the automated AST system at a rate of up to 4 plates every 8, 10 or 12 minutes.

26. The method of claim 11, wherein, on average, no more than 8 hours elapses between the step of inserting an AST cassette into the system and the step of reporting the QSR or MIC to a user of the automated AST system for the same AST cassette.

27. The method of claim 26, wherein the average time elapsed is no more than 6 hours.
